(19) United States
(12) Patent Application Publication
(10) Pub. No.: US 2009/0149954 A1
(43) Pub. Date: Jun. 11, 2009

(54) Bone substitute

(76) Inventors: Xianbo Hu, Alpharetta, GA (US); Thomas Hirt, Rebstein (CH); Xiaowen Zhai, Alpharetta, GA (US); Sameer Shums, Lilburn, GA (US)

Correspondence Address: LAW OFFICE OF COLLEN A. BEARD, LLC, P. O. BOX 1064, DECATUR, GA, 30031-1064, US

(21) Appl. No.: 12/315,659

(22) Filed: Dec. 5, 2008

Related U.S. Application Data
(60) Provisional application No. 61/005,716, filed on Dec. 7, 2007.

(51) Int. Cl. A61F 2/28

(52) U.S. Cl. 623/16.11; 623/23.61

(57) ABSTRACT

A bone substitute suitable for both load bearing and non load bearing applications having an aqueous phase, preferably a hydrogel phase formed by macromers, a low water content (hydrophobic) phase formed by amphiphilic monomers, and an inorganic filler.

### CROSS REFERENCE TO RELATED APPLICATIONS

The present application is related to and claims priority to U.S. Provisional Application Ser. No. 61/005,716 filed on Dec. 7, 2007, the entire contents of which are incorporated herein by reference.

### BACKGROUND OF THE INVENTION

The invention relates to a composition for forming a bone substitute, a bone substitute, and method of making the bone substitute in vivo. More particularly, the present invention relates to a composition for forming a bone substitute having the capability of setting in vivo very quickly. The bone substitute comprises an aqueous phase, preferably a hydrogel phase formed by crosslinking one or more macromers, an amphiphilic monomer, and an inorganic filler. The relative amounts of each component in the composition can be optimized in order to form bone substitutes useful with bones having different mechanical characteristics.

When cancellous bone becomes diseased it is more prone to compression fracture or collapse. For example, in the cases of osteoporosis, avascular necrosis, and cancer, the cancellous bone no longer provides interior support for the cortical bone. Other cases when a bone substitute is useful involve infected bone, poorly healing bone, or bone fractured by severe trauma. These conditions, if not successfully treated, can result in deformities, chronic complications, and an overall adverse impact upon the quality of life. Therefore, it becomes necessary to fix cancellous bone with a prosthesis. For example, a spinal vertebra may become damaged due to trauma or disease resulting in a collapse or misalignment of the vertebrae. A prosthesis should fill in degenerated/diseased cancellous bone and mechanically stabilize the vertebrae whose structural integrity may have been compromised. Preferably the prosthesis will be injectable and will mimic the characteristics and function of the cancellous bone that it is replacing. Materials used in this type of prosthesis are commonly called bone void filler, bone substitute, or bone cement. The term “bone substitute” as used herein includes bone void filler, bone cement, and bone substitute.

A wide range of materials have been used as bone substitutes. However, most of the commercially available bone cements for the fixation of artificial joints and vertebral fractures are chemically based on the same material, polymethylmethacrylate and its derivatives. PMMA has been used for more than 50 years in joint reconstruction and is popular in joint replacement prostheses such as total hip and total knee prostheses. PMMA bone cements used in vertebroplasty or kyphoplasty can relieve patients' pain and stabilize the spine very effectively in a short term.

However, there are complications associated with the use of PMMA for both vertebroplasty or kyphoplasty such as: death due to sudden blood pressure drop related to the release of MMA monomer into the vascular system; material extravasation into spinal canal leading to neurologic deficit due to the compression of the spinal cord and/or nerve roots; new fracture of adjacent non-augmented vertebrae; and pulmonary embolism of the PMMA. Most of these complications are associated with the intrinsic characteristics of PMMA based bone cement such as exotherm of the free radical polymerization, release of MMA monomer, the high stiffness of PMMA, and its lack of adhesion to bone.

The generation of heat to the surrounding tissue as PMMA bone cement cures is a matter of record. Polymerization temperatures range from 40 C to 120 C. Various researchers have shown that the interface temperature for PMMA bone cement could reach as high as 100-120 C; the maximum temperature generated was 107 C for a cement thickness of 10 mm and 60 C for a thickness of 3 mm; and that the maximum temperature decreased to 41 C if the thickness was only 1 mm.

PMMA has been modified by several researchers to address its limitations. Modifications include the incorporation of bioactive fillers to improve the mechanical properties and increase the adhesion to bone; the development of novel formulations with higher ductility and lower tensile modulus to produce a more even stress distribution between a total joint replacement prosthesis and the bone; the modification of the liquid phase in order to decrease the thermal and chemical damage to tissue; and the development of bone cements that contain a hydrogel forming monomer, e.g. acrylic acid or 2-hydroxyethyl methacrylate, in order to adjust the mechanical and swelling properties of the bone cement system.

These modifications have been made to improve the function of bone cements for fixing a total joint replacement prosthesis to bone. Unfortunately, these modifications do not address the additional limitation of the mechanical mismatch between rigid PMMA-based bone cement and spongy osteoporototic cancellous bone. The stresses placed upon bone cement in the spine are generally compressive and much lower in magnitude than cement used in total joint replacement procedures. These stresses in the vertebral body are estimated to be approximately 1 MPa or less, which is 10 times less than the tensile strength and 1,000 times less than the compressive modulus of commercial bone cements. There is need for a more biocompatible bone cement from both a scientific and industrial point of view.

Calcium phosphate bone substitutes (CPBS), including ceramics prepared by sintering (thermal consolidation) and cements prepared by precipitating from aqueous solution around room temperature or at body temperature, have been developed and used for filling of bone defects due to their good biocompatibility, bioactivity, and degradability. Due to its capability of setting under ambient conditions, calcium phosphate cement has attracted much attention as a reconstructive material for bony defects since it was developed in the 1980's. However CPBS, specifically calcium phosphate bone cement, has intrinsic limitations. For one thing, its relatively low mechanical properties limit its clinical applications. Most CPBSs consist of an aqueous solution and a powder containing one or more solid calcium and/or phosphate salts. The liquid and powder are mixed together to make a paste that is implanted or injected, and that sets at room or body temperature by precipitation of one or more different solid compounds, mainly hydroxyapatite. Since the mechanical properties are controlled by the entanglement or interlocking of the precipitated crystals within the paste, CPBS is not good for load bearing bone substitute applications.

Ceramic bone substitutes have other issues such as they easily migrate or disperse into blood and surrounding tissues, their difficulty in completely fitting the bone surface, inadequate setting time, cohesion, they take a long time to reach their ultimate compressive strength after setting, and the degradation rate of the cement in vivo.

The setting time of most CPBSs is in the range of 5 to 45 minutes at 37 C in vitro. The setting time in vivo usually is longer than that in vitro since ions in the body fluid such as Mg⁺, and organic compounds can inhibit the setting reaction. They also tend to disintegrate upon early contact with blood or other aqueous (body) fluid flow right after injection. Once the paste decays, due to body fluid penetration into the paste, it will not set. This will result in complications in clinical applications. There are even more concerns about CPBS than PMMA cement for vertebral body augmentation since pulmonary embolism of CPBS results in more severe cardiovascular deterioration due to coagulation activation. For example, CPBS tends to disintegrate and break into smaller pieces more easily than PMMA in lung.

For these reasons, cohesion or cohesiveness (defined as the resistance to washout of the formulation for in vivo polymerizing or setting device) of CPBS must be improved. It also should be taken into consideration that setting time will depend on all factors involved in the cement manufacture such as liquid/powder ratio, content/crystallinity/size of the seed material, amount of accelerator in the liquid phase, particle size of the reactants, and temperature. What seems clear from practical experience with CPBSs is that there is a strong correlation between setting time and mechanical properties of the substitute. This means that when a cement is designed to minimize the setting time, then the compressive strength is also at a minimum. On the other hand, when the same formulation is designed to obtain maximum compressive strength, then the setting time is increased. A compromise between these properties is therefore necessary to ensure a cement suitable for clinical application.

The compressive strength of CPBS increases as a function of time after setting in vivo. It reaches the designed or ultimate strength in a broad range of time such as from at least hours to about days with immersion in Ringer's solution at 37 C. In most clinical applications, bone substitutes contact with human cancellous bone directly and a mechanical requirement such as strength and modulus must be at least as high as that of human cancellous bone in a time as short as possible to allow load bearing and/or build up stability. This is a need for fast-setting CPBSs for a shorter hospitalization and shorter period of inactivity. The high rate of early return to normal daily activities is especially appealing for both patient and physician. It is preferred to have a bone substitute that obtains its final or ultimate mechanical properties right after setting, and the property is independent of setting time.

Different approaches have been used to improve the cohesion or cohesiveness, or shorten the setting time of calcium phosphate bone substitutes such as adding sodium alginate which produces a significant delay in the setting reaction and decreases the mechanical properties in vitro and adding accelerator NaH₂PO₄.2H₂O or Na₂HPO₄ in the liquid part, or adding dicalcium phosphate and/or calcium carbonate to the powder part. Cellulose derivatives such as hydroxylpropyl methylcellulose (HMPC), carboxyl methylcellulose (CMC), chitosan, chitosan lactate, and sodium hyaluronate have been used to improve the cohesion or time to resistant to washout dramatically. However, these either increase the setting time or decrease the mechanical properties.

Combining polymers and CPBS appears an attractive approach to improve the mechanical properties of CPBS. A wide range of polymers including poly(lactic acid) (PLA), poly(lactic-co-glycolic acid) (PLGA), gelatin, poly(vinyl alcohol) (PVA), sodium alginate and sodium polyacrylate, and polyelectrolytes/poly(ethylene oxide)/poly(ethylenimine)/poly(sodium 4-styrenesulfonate) have been explored and markable increases in mechanical properties were found, but with unacceptable reduction in workability and setting time.

U.S. Pat. No. 6,652,883 and U.S. Patent Publication 2005/0288789, both to BioCure, Inc., disclose compositions useful for tissue bulking and replacement or augmentation of a spinal disc nucleus pulposus that are based on a hydrogel made from macromers having a backbone of units with a 1,2-diol and/or 1,3-diol structure, optionally including an amphiphilic comonomer. Such polymers include poly(vinyl alcohol) (PVA) macromers and hydrolyzed copolymers of vinyl acetate, for example, copolymers with vinyl chloride or N-vinylpyrrolidone. The PVA backbone polymer contains pendant chains bearing crosslinkable groups and, optionally, other modifiers. The macromers form a hydrogel when crosslinked. This composition can be injected as a liquid and crosslinked into a solid hydrogel in vivo. The hydrogel is suitable for many bio-applications. However, a composition useful as a bone cement should ideally have particular properties not provided by the hydrogels taught in these publications.

The mechanical properties of human cancellous bone vary significantly across individuals depending on their age, sex, and disease, and also across anatomic sites such as lumbar, tibia, or femur. The average elastic modulus of vertebral cancellous bones for healthy people falls in a broad range, from to 67±45 MPa for ages 15 to 87, to lower values of 22.8±15.5 MPa for ages 71 to 84. The strength, most often characterized by the parameter ultimate stress, is about 0.5 to 5 MPa, about one-hundredth of its elastic modulus. For vertebral cancellous bones, the ultimate strain in both compression and tension is very similar up to 5%. For individuals with osteoporosis disease, the mechanical properties of the vertebral cancellous bone are lower than those of healthy people. Therefore, the mechanical properties of a bone cement formulation should be adjustable and cover a wide range of tangent modulus as well as strength. Toughness is also important for safety concerns due to the long-term complex stress environment and debris formation close to the central nerve system.

### SUMMARY OF THE INVENTION

The invention relates to a bone substitute having an aqueous phase, a low water content (hydrophobic) phase, and an inorganic filler. The aqueous phase is preferably a hydrogel phase and is provided by crosslinking one or more crosslinkers, preferably macromers. The low water content phase is provided by an amphiphilic monomer or oligomer. The amphiphilic monomer ties together the aqueous phase and the inorganic filler, adding toughness. The composition for forming the bone substitute is capable of fast setting in body fluid in vivo and can be formulated to yield a bone substitute with mechanical properties matching those of different anatomic bones with different mechanical requirements. The bone substitute is suitable for both load bearing and non load bearing applications. The bone substitute can be used to fix bone voids and bone fractures, more particularly to treat porous bone such as osteoporotic bone.

Setting of the bone substitute is controlled by crosslinking the one or more crosslinkers rather than the in vivo setting mechanism of calcium salts; therefore, the working time is adjustable by changing the crosslinking speed, such as by changing the concentration of initiating agent. Hydrogel macromers (e.g. PVA macromers, polyHEMA macromers, PEG macromers, PEI macromers) and the amphiphilic monomer provide excellent affinity to the calcium salts. The bone substitute has an excellent combination of mechanical properties and deliverability. The bone substitute provides cohesiveness and mechanical properties including strength, toughness, and integrity, especially under complex loading situations. Calcium salts present at the surface can also have osteoconductive and osteoinductive properties.

The tangent modulus of the novel bone substitute is in a range of 5 to 800 MPa, preferably about 50 to 500 MPa at 1% strain. Tangent modulus is the slope of the compression stress-strain curve at any specified stress or strain. The ultimate stress is in a range between about 0.5 to 30 MPa, preferably about 5 to 30 MPa. The novel bone substitute shows very good thermal and dimensional stability in simulated body fluid, Ringer's solution at 37 C and 70 C. The tangent modulus, ultimate stress, and ultimate strain are very constant over time at both temperatures and the material did not change its dimension over time at both temperatures. The bone substitute has a low exothermic temperature during polymerization, and a very good cohesiveness. In one embodiment it can be delivered through a long fine needle, e.g. a 4 to 6 inch long 18 gauge needle, and reach the final mechanical properties within half an hour after delivery.

### BRIEF DESCRIPTION OF THE FIGURE

FIG. 1 is a diagram of stress versus strain for compact bone (1), PMMA (2), cancellous (trabecular) bone (3), and three formulations of the inventive bone substitute (4-7). Line 4 represents bone substitute with crosslinker and inorganic filler. Line 5 represents bone substitute with inorganic filler and lower content of crosslinker. Line 6 represents bone substitute with inorganic filler only (no crosslinker). Line 7 represents bone substitute without filler or crosslinker.

### DETAILED DESCRIPTION OF THE INVENTION

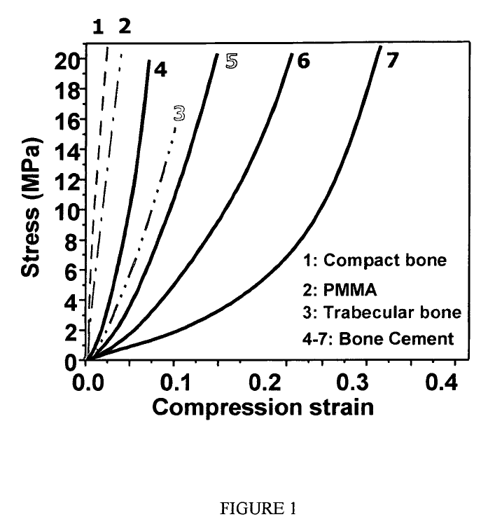

The invention is a bone substitute that has an aqueous phase, a low water content (hydrophobic) phase provided by an amphiphilic monomer or oligomer, and an inorganic filler. The aqueous phase is preferably a hydrogel phase that is made from one or more crosslinkers, preferably macromers. The components form a phase separated system, where the aqueous or hydrogel phase provides a natural response to the initial load of a slow increase of stress at initial strains (“soft response”). Another way to describe soft response is that the bone substitute shows a slow onset of stress with increasing compressive strain. This is illustrated in FIG. 1, by the initial slope and shape of the strain versus stress curve for the bone substitutes. The monomer provides a hydrophobic phase and provides the toughness required of the bone substitute, and the inorganic filler and any additional crosslinkers increase the modulus and strength of the bone substitute. The inorganic filler can also increase the biocompatibility and potential bioactivity. In combination, a bone substitute is provided which can be formulated to have mechanical properties matching those of different anatomic bones. The bone substitute can be used for fixation of cancellous bone such as bone voids (non load bearing) or osteoporotic bone (load bearing) such as found in the spine.

The bone substitute formed in vivo conforms in shape to the space into which it is injected. The bone substitute has a tangent modulus of approximately up to 800 MPa at 1% strain (preferably about 50 to 500 MPa), from about 5-30 MPa at ultimate strain of 20-50%, and has the ability to withstand cyclic loading under physiologic conditions. The ultimate stress is in a range between about 0.5 to 30 MPa, preferably about 5 to 30 MPa. It may be advantageous for the bone substitute to swell upon implantation to fill the fracture space. Additional potential design features include adhesion to the native tissue. Tailoring of mechanical properties to match those of cancellous bones with different mechanical requirements is done by using crosslinker with variable molecular weight and crosslinks per chain. In addition, mechanical properties can be altered by using different crosslinkers, different types and amount of monomer, and different amounts and types of inorganic filler. The crosslinkers, monomers, inorganic fillers, initiators, and other additives are described in detail below.

Aqueous Phase

The aqueous phase forms about 10 to 40% by volume of the bone substitute. In one embodiment the aqueous phase is formed without addition of a crosslinking macromer, using simply amphiphilic monomer. Most desirably, the aqueous phase is a hydrogel phase formed from one or more hydrogel forming macromers (macromolecular monomers) and water. When the aqueous phase is formed by hydrogel forming macromers, they are present up to about 10 volume percent.

Macromers include those made from polyvinyl alcohol (PVA) (described further below); polyethyleneimine (PEI); poly(hydroxyalkyl methacrylates, e.g. poly(hydroxyethyl methacrylate) (PHEMA); poly(hydroxyalkylacrylates, e.g. poly(hydroxyethylacrylate) (HEA); polyethylene glycol (PEG); polypropylene glycol (PPG); poly(2-alkyl-oxazolines); polyvinylpyrrolidone (PVP); polyacrylamides, e.g. poly(dimethylacrylamide) (PDMA) or copolymers with different acrylates; dendrimers from, e.g., PEG, PHEMA, PDMA. The macromers can be linear, branched, hyperbranched, or have a dendritic structures. It can be a homo- or copolymer that is water-soluble.

PVA Based Macromers

The PVA macromers have a backbone of a polymer comprising units with a 1,2-diol and/or 1,3-diol structure and at least two pendant chains including a crosslinkable group. The macromer backbone can optionally have other pendant chains containing modifiers.

Polyvinyl alcohols (PVAs) that can be used as the macromer backbone include commercially available PVAs, for example Vinol® 107 from Air Products (MW 22,000 to 31,000, 98 to 98.8% hydrolyzed), Polysciences 4397 (MW 25,000, 98.5% hydrolyzed), BF 14 from Chan Chun, Elvanol® 90-50 from DuPont and UF-120 from Unitika. Other producers are, for example, Nippon Gohsei (Gohsenol®), Monsanto (Gelvatol®), Wacker (Polyviol®), Kuraray, Deriki, and Shin-Etsu. In some cases it is advantageous to use Mowiol® products from Hoechst, in particular those of the 3-83, 4-88, 4-98, 6-88, 6-98, 8-88, 8-98, 10-98, 20-98, 26-88, and 40-88 types.

It is also possible to use copolymers of hydrolyzed or partially hydrolyzed vinyl acetate, which are obtainable, for example, as hydrolyzed ethylene-vinyl acetate (EVA), or vinyl chloride-vinyl acetate, N-vinylpyrrolidone-vinyl acetate, and maleic anhydride-vinyl acetate. If the macromer backbones are, for example, copolymers of vinyl acetate and vinylpyrrolidone, it is again possible to use commercially available copolymers, for example the commercial products available under the name Luviskole® from BASF. Particular examples are Luviskol VA 37 HM, Luviskol VA 37 E and Luviskol VA 28. If the macromer backbones are polyvinyl acetates, Mowilith 30 from Hoechst is particularly suitable.

The PVA preferably has a molecular weight of at least about 2,000. As an upper limit, the PVA may have a molecular weight of up to 1,000,000. Preferably, the PVA has a molecular weight of up to about 130,000, more preferably up to about 60,000.

The PVA usually has a poly(2-hydroxy)ethylene structure. The PVA may also include hydroxy groups in the form of 1,2-glycols. The PVA can be a fully hydrolyzed PVA, with all repeating groups being—CH₂—CH(OH), or a partially hydrolyzed PVA with varying proportions (1% to 25%) of pendant ester groups. PVA with pendant ester groups have repeating groups of the structure CH₂—CH(OR) where R is COCH₃ group or longer alkyls, as long as the water solubility of the PVA is preserved. The ester groups can also be substituted by acetaldehyde or butyraldehyde acetals that impart a certain degree of hydrophobicity and strength to the PVA. For an application that requires an oxidatively stable PVA, the commercially available PVA can be broken down by NaIO₄—KMnO₄ oxidation to yield a small molecular weight (2000 to 4000) PVA.

The PVA is prepared by basic or acidic, partial or virtually complete, hydrolysis of polyvinyl acetate. In a preferred embodiment, the PVA comprises less than 50% acetate units, especially less than about 25% of acetate units. Preferred amounts of residual acetate units in the PVA, based on the sum of alcohol units and acetate units, are approximately from 3 to 25%.

The macromers have at least two pendant chains containing groups that can be crosslinked. Group is defined herein to include single polymerizable moieties, such as acrylates, as well as larger crosslinkable regions, such as oligomeric or polymeric regions. The crosslinkers are desirably present in an amount of from approximately 0.01 to 15 milliequivalents of crosslinker per gram of backbone (meq/g), more desirably about 0.05 to 5 milliequivalents per gram (meq/g). The macromers can contain more than one type of crosslinkable group.

The pendant chains are attached via the hydroxyl groups of the backbone. Desirably, the pendant chains having crosslinkable groups are attached via cyclic acetal linkages to the 1,2-diol or 1,3-diol hydroxyl groups. Desirable crosslinkable groups include (meth)acrylamide, (meth)acrylate, styryl, vinyl ester, vinyl ketone, vinyl ethers, etc. Particularly desirable are ethylenically unsaturated functional groups. A particularly desirable crosslinker is N-acryloyl-aminoacetaldehyde dimethylacetal (NAAADA) in an amount from about 1 to 1,750 crosslinkers per macromer.

Specific macromers that are suitable for use in the compositions are disclosed in U.S. Pat. Nos. 5,508,317, 5,665,840, 5,807,927, 5,849,841, 5,932,674, 5,939,489, and 6,011,077.

In one embodiment, units containing a crosslinkable group conform, in particular, to the formula I

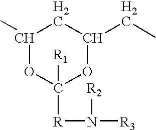

in which R is a linear or branched C₁-C₈ alkylene or a linear or branched C₁-C₁₂ alkane. Suitable alkylene examples include octylene, hexylene, pentylene, butylene, propylene, ethylene, methylene, 2-propylene, 2-butylene and 3-pentylene. Preferably lower alkylene R has up to 6 and especially preferably up to 4 carbon atoms. The groups ethylene and butylene are especially preferred. Alkanes include, in particular, methane, ethane, n- or isopropane, n-, sec- or tert-butane, n- or isopentane, hexane, heptane, or octane. Preferred groups contain one to four carbon atoms, in particular one carbon atom.

R₁ is hydrogen, a C₁-C₆ alkyl, or a cycloalkyl, for example, methyl, ethyl, propyl or butyl and R₂ is hydrogen or a C₁-C₆ alkyl, for example, methyl, ethyl, propyl or butyl. R₁ and R₂ are preferably each hydrogen.

R₃ is an olefinically unsaturated electron attracting copolymerizable radical having up to 25 carbon atoms. In one embodiment, R₃ has the structure

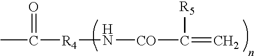

where R₄ is the

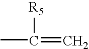

group if n=zero, or the

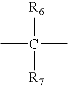

bridge if n=1;

R₅ is hydrogen or C₁-C₄ alkyl, for example, n-butyl, n- or isopropyl, ethyl, or methyl;

n is zero or 1, preferably zero; and

R₆ and R₇, independently of one another, are hydrogen, a linear or branched C₁-C₈ alkyl, aryl or cyclohexyl, for example one of the following: octyl, hexyl, pentyl, butyl, propyl, ethyl, methyl, 2-propyl, 2-butyl or 3-pentyl. R₆ is preferably hydrogen or the CH₃ group, and R₇ is preferably a C₁-C₄ alkyl group. R₆ and R₇ as aryl are preferably phenyl.

In another embodiment, R₃ is an olefinically unsaturated acyl group of formula R₈—CO—, in which R₈ is an olefinically unsaturated copolymerizable group having from 2 to 24 carbon atoms, preferably from 2 to 8 carbon atoms, especially preferably from 2 to 4 carbon atoms. The olefinically unsaturated copolymerizable radical R₈ having from 2 to 24 carbon atoms is preferably alkenyl having from 2 to 24 carbon atoms, especially alkenyl having from 2 to 8 carbon atoms and especially preferably alkenyl having from 2 to 4 carbon atoms, for example ethenyl, 2-propenyl, 3-propenyl, 2-butenyl, hexenyl, octenyl or dodecenyl. The groups ethenyl and 2-propenyl are preferred, so that the group —CO—R₈ is the acyl radical of acrylic or methacrylic acid.

In another embodiment, the group R₃ is a radical of formula

—[CO—NH—(R₉—NH—CO—O)_{q}—R₁₀—O]ₚ—CO—R₈

wherein p and q are zero or one and

R₉ and R₁₀ are each independently lower alkylene having from 2 to 8 carbon atoms, arylene having from 6 to 12 carbon atoms, a saturated divalent cycloaliphatic group having from 6 to 10 carbon atoms, arylenealkylene or alkylenearylene having from 7 to 14 carbon atoms or arylenealkylenearylene having from 13 to 16 carbon atoms, and

R₈ is as defined above.

Lower alkylene R₉ or R₁₀ preferably has from 2 to 6 carbon atoms and is especially straight-chained. Suitable examples include propylene, butylene, hexylene, dimethylethylene and, especially preferably, ethylene.

Arylene R₉ or R₁₀ is preferably phenylene that is unsubstituted or is substituted by lower alkyl or lower alkoxy, especially 1,3-phenylene or 1,4-phenylene or methyl-1,4-phenylene.

A saturated divalent cycloaliphatic group R₉ or R₁₀ is preferably cyclohexylene or cyclohexylene-lower alkylene, for example cyclohexylenemethylene, that is unsubstituted or is substituted by one or more methyl groups, such as, for example, trimethylcyclohexylenemethylene, for example the divalent isophorone radical.

The arylene unit of alkylenearylene or arylenealkylene R₉ or R₁₀ is preferably phenylene, unsubstituted or substituted by lower alkyl or lower alkoxy, and the alkylene unit thereof is preferably lower alkylene, such as methylene or ethylene, especially methylene. Such radicals R₉ or R₁₀ are therefore preferably phenylenemethylene or methylenephenylene.

Arylenealkylenearylene R₉ or R₁₀ is preferably phenylene-lower alkylene-phenylene having up to 4 carbon atoms in the alkylene unit, for example phenyleneethylenephenylene.

The groups R₉ and R₁₀ are each independently preferably lower alkylene having from 2 to 6 carbon atoms, phenylene, unsubstituted or substituted by lower alkyl, cyclohexylene or cyclohexylene-lower alkylene, unsubstituted or substituted by lower alkyl, phenylene-lower alkylene, lower alkylene-phenylene or phenylene-lower alkylene-phenylene.

The group —R₉—NH—CO—O— is present when q is one and absent when q is zero. Macromers in which q is zero are preferred.

The group —CO—NH—(R₉—NH—CO—O)_{q}—R₁₀—O is present when p is one and absent when p is zero. Macromers in which p is zero are preferred.

In macromers in which p is one, q is preferably zero. Macromers in which p is one, q is zero, and R₁₀ is lower alkylene are especially preferred.

All of the above groups can be monosubstituted or polysubstituted, examples of suitable substituents being the following: C₁-C₄ alkyl, such as methyl, ethyl or propyl, —COOH, —OH, —SH, C₁-C₄ alkoxy (such as methoxy, ethoxy, propoxy, butoxy, or isobutoxy), —NO₂, —NH₂, —NH(C₁-C₄), —NH—CO—NH₂, —N(C₁-C₄ alkyl)₂, phenyl (unsubstituted or substituted by, for example, —OH or halogen, such as Cl, Br or especially I), —S(C₁-C₄ alkyl), a 5- or 6-membered heterocyclic ring, such as, in particular, indole or imidazole, —NH—C(NH)—NH₂, phenoxyphenyl (unsubstituted or substituted by, for example, —OH or halogen, such as Cl, Br or especially I), an olefinic group, such as ethylene or vinyl, and CO—NH—C(NH)—NH₂.

Preferred substituents are lower alkyl, which here, as elsewhere in this description, is preferably C₁-C₄ alkyl, C₁-C₄ alkoxy, COOH, SH, —NH₂, —NH(C₁-C₄ alkyl), —N(C₁-C₄ alkyl)₂ or halogen. Particular preference is given to C₁-C₄ alkyl, C₁-C₄ alkoxy, COOH and SH.

For the purposes of this invention, cycloalkyl is, in particular, cycloalkyl, and aryl is, in particular, phenyl, unsubstituted or substituted as described above.

A particularly preferred macromer has a PVA backbone (14 kDa, 17% acetate incorporation) modified with 1.07 meq/g N-acrylamidoacetaldehyde dimethyl acetal (NAAADA) pendant polymerizable groups (about 15 crosslinks per chain). In some preferred embodiments the PVA backbone is also modified with a hydrophobic modifier acetaldehyde diethyl acetal (AADA) present in an amount from about 0 to 4 milliequivalents per gram (meq/g) of PVA.

Additional Crosslinkers

Additional crosslinkers, as used herein, means small molecules which have at least two reactive groups. The crosslinkers can react with the macromer, monomer, and/or themselves. More specifically, crosslinkers are small molecules with at least 2 acrylate, acrylamide, epoxy, thiol, or amide groups. The crosslinker can be either water soluble or water insoluble. Examples of crosslinkers include but are not limited to methylenebisacrylamide (MBA), N,N′-(1,2-dihydroxyethylene)bis-acrylamide (DHEBA), N,N′-ethylenebis(acrylamide), methylenebismethacrylamide (MBMA) N,N′-hexamethylenebisacrylamide, N,N′-hexamethylenebismethacrylamide, N,N′-Bis(acryloyl)cystamine, ethidium bromide-N,N′-bismethacrylamide, 1,4-Bis(acryloyl)piperazine, ethylene glycol dimethacrylate, glycerol dimethacrylate (GD), glycerol 1,3-diglycerolate diacrylate, bisphenol A ethoxylate diacrylate (BAED), tetra(ethyleneglycol) diacrylate (TEGD), pentaerythritol triacrylate, trimethylolpropane triacrylate (TT), bisphenol A dimethacrylate (BADM), pentaerythritol tetraacrylate (PTA), trimethylolpropane trimethacrylate (TMPTMA), 1,6-hexanediol di(meth)acrylate, divinyl benzene, triallylamine, epoxided soybean oil (ESO), acrylate epoxided soybean oil, and acrylamided epoxided soybean oil. The molecular weight of the crosslinkers is from 30 to 5000, preferably, 30 to 1000. The volume percent of the crosslinker is from 0 to 30, preferably 2 to 15.

Amphiphilic Monomers

As used herein, the term amphiphilic means that one portion of the molecule is hydrophilic and one portion of the molecule is hydrophobic. The term amphiphilic monomer also refers to amphiphilic oligomers. In one embodiment, the hydrophilic portion is water soluble and the hydrophobic portion is not water soluble. The monomer as a whole is preferably wholly or partially water soluble. Examples of useful amphiphilic monomers are diacetone acrylamide (DAA), N-vinyl caprolactam, N-(butoxymethyl)acrylamide, N-acroyl morpholine, crotonamide, N,N-dimethyl acrylamide, N-octadecylacrylamide, and acrylamide.

Because the amphiphilic monomers are water soluble, they provide a mixing media and low viscosity for the paste, which can be beneficial to the delivery of the paste through an 18 gauge needle. When the amphiphilic monomers are copolymerized with the macromers described above, or even polymerized by themselves, a product results that is more cohesive and has higher compressive strength than a product not containing the amphiphilic monomer. Often the water soluble amphiphilic monomers form polymers with low water solubility. Desirably, the monomer is included in an amount ranging from about 5 to 80 volume percent, most preferably about 20 to 60 volume percent (where volume percent is the percent by volume of the total paste). In a preferred embodiment, the amphiphilic monomer is DAA in an amount from about 20 to 70 volume percent.

Inorganic Fillers

The inorganic filler can be selected from many different types of metal oxides such as alumina, silica, and zirconia, and non-oxides such as carbides and nitrides, calcium salts, natural and synthetic clays, bioactive glasses, or a mixture of above. The inorganic filler preferably is a calcium salt, and more preferably is a calcium phosphate such as monocalcium phosphate monohydrate (MCPM), dicalcium phosphate (DCP), tricalcium phosphate (TCP), amorphous calcium phosphate (ACP), hydroxyapatite (HA), tetracalcium phosphate (tetCP), or a combination of the above. The calcium phosphates are either natural or synthetic. The calcium phosphates are either bioresorbable or non-resorbable. The calcium phosphates maintain their particulate nature in the composite. Additionally, the calcium:phosphate ratio in the calcium phosphates may vary, in the range of 0.5 to 2.0. The calcium phosphates are prepared via precipitation from aqueous solution at or around room temperature or by a thermal reaction at high temperature as described in U.S. Pat. No. 7,393,405; or Bohner, Injury, International Journal of The Care of the Injured, 2000, 31, S-D37; or Bohner, Biomaterials, 2005, 26, 6423. The calcium salts may include other salts of calcium, such as calcium sulfate, calcium carbonate as well as a combination of these and/or the above calcium phosphates.

Calcium phosphates having different sizes and/or crystallinity can be used to affect the mechanical properties of the bone substitute. Preferably the calcium phosphate has a size from about 0.001 to 500 μm, more preferably 0.01 to 100 μm. Different calcium phosphates may also affect the biocompatibility and bioactivity of the bone substitute. The inorganic filler is present in an amount ranging from about 10 to 40 volume percent, most preferably about 15 to 30 volume percent.

Initiators

The ethylenically unsaturated groups of the macromer and/or amphiphilic monomer can be polymerized via free radical initiated polymerization, including with initiation via photoinitiation, redox initiation, and thermal initiation. Systems employing these means of initiation are well known to those skilled in the art and may be used in the compositions taught herein. The desired amounts of the initiator components will be determined by concerns related to gelation speed or setting time, toxicity, extent of gelation desired, and stability. The initiation system provides a bone substitute with both an adjustable setting time and a fast setting without sacrificing mechanical properties.

In one embodiment, a two part redox system is employed. One part of the system contains a reducing agent. Examples of reducing agents are ferrous salts (such as ferrous gluconate dihydrate, ferrous lactate dihydrate, or ferrous acetate), cuprous salts, cerous salts, cobaltous salts, permanganate, manganous salts, and tertiary amines such as N,N,N,N-tetramethylethylene diamine (TMEDA). The other half of the solution contains an oxidizing agent such as hydrogen peroxide, t-butyl hydroperoxide, t-butyl peroxide, benzoyl peroxide, cumyl peroxide, potassium persulfate, or ammonium persulfate.

Either or both of the redox solutions can contain macromer, or it may be in a third solution. The solutions containing reductant and oxidant are combined to initiate the polymerization. It may be desirable to use a coreductant such as ascorbate, for example, to recycle the reductant and reduce the amount needed.

Modifier Groups

The macromers can include further modifier groups and reactive groups. Some such groups are described in U.S. Pat. Nos. 5,508,317, 5,665,840, 5,807,927, 5,849,841, 5,932,674, 5,939,489, and 6,011,077 and include hydrophobic modifiers such as acetaldehyde diethyl acetal (AADA), butyraldehyde, and acetaldehyde or hydrophilic modifiers such as N-(2,2-dimethoxy-ethyl) succinamic acid, amino acetaldehyde dimethyl acetal, and aminobutyraldehyde dimethyl acetal. These groups may be attached to the macromer backbone, or to other monomeric units included in the backbone. Reactive groups and optional modifier groups can be bonded to the macromer backbone in various ways, for example through a certain percentage of the 1,3-diol units being modified to give a 1,3-dioxane, which contains a crosslinkable group, or a further modifier, in the 2-position. Modifiers include those to modify the hydrophobicity or hydrophilicity, active agents or groups to allow attachment of active agents, photoinitiators, modifiers to enhance or reduce adhesiveness, modifiers to enhance the cohesion or cohesiveness of the paste before setting, modifiers to impart thermoresponsiveness, modifiers to impart other types of responsiveness, and additional reactive groups.

Attaching a cellular adhesion promoter to the macromers can enhance cellular attachment or adhesiveness of the composition. These agents are well known to those skilled in the art and include carboxymethyl dextran, proteoglycans, collagen, gelatin, glucosaminoglycans, fibronectin, lectins, polycations, and natural or synthetic biological cell adhesion agents such as RGD peptides.

Having pendant ester groups that are substituted by acetaldehyde or butyraldehyde acetals, for example, can increase the hydrophobicity of the macromers and the formed hydrogel. One particularly useful hydrophobic modifying group is acetaldehyde diethyl acetal (AADA) present in an amount from about 0 to 4 milliequivalents per gram (meq/g) of PVA.

Hydrophilic modifiers such as —COOH in the form of N-(2,2-dimethoxy-ethyl) succinamic acid in an amount from about 0 to 2 meq/g PVA can be added to the hydrogel to enhance performance of the hydrogel, such as favorable energetic interactions between the hydrogel and the inorganic filler.

It may also be desirable to include on the macromer a molecule that allows visualization of the formed hydrogel. Examples include dyes and molecules visualizable by magnetic resonance imaging.

Contrast Agents

The bone substitute can be made containing a contrast agent. A contrast agent is a biocompatible material capable of being monitored by, for example, radiography. The contrast agent can be water soluble or water insoluble. Examples of water soluble contrast agents include metrizamide, iopamidol, iothalamate sodium, iodomide sodium, and meglumine. Iodinated liquid contrast agents include Omnipaque®, Visipaque®, and Hypaque-76®. Examples of water insoluble contrast agents are tantalum, tantalum oxide, barium sulfate, zirconium oxide (zirconia), gold, tungsten, platinum, and calcium phosphate. These are commonly available as particles preferably having a size of about 10 μm or less. Coated fibers, such as tantalum-coated Dacron fibers can also be used.

Active Agents

The bone substitute can include an effective amount of one or more biologically or structurally active agents. It may be desirable to deliver the active agent from the bone substitute. Active agents that it may be desirable to deliver include prophylactic, therapeutic, diagnostic, and structural agents including organic and inorganic molecules (collectively referred to herein as an “active agent” or “drug”). A wide variety of active agents can be incorporated into the bone substitute. Release of the incorporated additive from the bone substitute is achieved by diffusion of the agent from the bone substitute, and/or degradation of a chemical link coupling the agent to the bone substitute. In this context, an “effective amount” refers to the amount of active agent required to obtain the desired effect.

Examples of active agents that can be incorporated include, but are not limited to, analgesics for the treatment of pain, for example ibuprofen, acetaminophen, and acetylsalicylic acid; antibiotics for the treatment of infection, for example tetracyclines, gentamycin, and penicillin and derivatives; and additives for the treatment of infection, for example silver ions, silver (metallic), and copper (metallic).

It may be advantageous to incorporate material of biological origin or biological material derived from synthetic methods of manufacture such as proteins, polypeptides, polysaccharides, proteoglycans, and growth factors.

It may be desirable to include additives to improve the swelling and space-filling properties of the prosthetic disc, for example, dehydrated spheres, fibers, etc., hydrophilic polymers, such AMPS, etc., or hydrocolloids, such as agar, alginates, carboxymethylcellulose, gelatin, guar gum, gum arabic, pectin, starch, and xanthum gum.

Other additives that may prove advantageous are additives to improve the adhesive properties of the prosthetic disc, including positively charged polymers, such as Quat, etc., PVA modified with positive-charged moieties attached to the backbone, chitosan, and mussel-based adhesives.

Incorporation of additives to improve the toughness properties of the injectable bone substitutes may prove desirable such as low modulus spheres, fibers, etc that act as “crack arrestors” and high modulus spheres, fibers, etc that act as “reinforcing” agents.

Active agents can be incorporated into the composition simply by mixing the agent with the composition prior to administration. The active agent will then be entrapped in the hydrogel that is formed upon administration of the composition. The active agent can be in compound form or can be in the form of degradable or nondegradable nano or microspheres. It some cases, it may be possible and desirable to attach the active agent to the macromer. The active agent may be released from the macromer or hydrogel over time or in response to an environmental condition.

It may be desirable to include a peroxide stabilizer in redox initiated systems. Examples of peroxide stabilizers are Dequest® products from Solutia Inc., such as for example Dequest® 2010 and Dequest® 2060S. These are phosphonates and chelants that offer stabilization of peroxide systems. Dequest® 2060S is diethylenetriamine penta(methylene phosphonic acid). These can be added in amounts as recommended by the manufacturer.

Methods of Using the Bone Substitute

To use the bone substitute, a paste is prepared by mixing the water, macromer and/or amphiphilic monomer, inorganic filler, and any other components such as an initiator, in the desired concentrations for each and proportion to each other. The paste may be prepared as a two-part composition, which form the hydrogel when mixed together. The macromer, inorganic filler, and monomer are desirably crosslinked into the bone substitute in vivo. Mixing can be by any mixing means known in the art.

After creation of a space in the bone, if desired, an effective amount of the paste composition is placed into the bone—preferably by a minimally invasive method. The term “effective amount”, as used herein, means the quantity of composition needed to fill the bone cavity. The composition may be administered over a number of treatment sessions. In one embodiment, the bone substitute can be used as a tissue adhesive in order to adhere bone tissue together.

In the preferred method of using the bone substitute, the paste composition is transferred into 1 to 10 ml Luer-lok syringe with care being taken to expel any air bubbles and then delivered using a needle of about 18 Gauge through the bone access port into the bone cavity under fluoroscopic guidance until the bone cavity has been filled to the desired level. In order to prevent the exposure of others to the radiation, a 10 to 16 inch long tubing can be used between the syringe and the needle. In the case of a two-part composition, the composition is mixed prior to injection in a syringe with an inner mixer and plunger or using a dual syringe method—transferring the mixture back and forth between two 10 ml syringes using a three way stopcock with care being taken to avoid air bubbles. In a powder/paste-liquid mixing system, the liquid is transferred to the powder/paste chamber, then the liquid-powder/paste is mixed with the mixing rod. The mixed paste composition will preferably crosslink into a formed hydrogel within 5 to 10 minutes post mixing.

The viscosity of the paste composition is, within wide limits, not critical, but the paste should preferably be injectable so that it can be delivered through an appropriately sized catheter or syringe needle. The viscosity will generally be controlled by the molecular weight of the macromers, the solids content of the paste, the content and size of the inorganic filler, and the type and amount of contrast agent present (if any) as well as the plasticizers, if desired. The solids content of the composition will preferably range from about 40 percent by weight to about 70 percent by weight, desirably from about 60 to 90 percent by weight. The composition is desirably deliverable through 8 gauge to 13 gauge 4-6 inch long cannula, preferably through even smaller needles such as 18 to 20 gauge 4 to 6 inch long needles.

In the preferred embodiment, the paste should be injected before substantial crosslinking has occurred for the right injectability. This procedure prevents blockage of the syringe needle or catheter with gelled polymer. In addition, in vivo crosslinking may allow anchoring of the hydrogel to host tissue by covalently bonding with collagen molecules present within the host tissue.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric.

Materials

Polyvinyl alcohol (PVA) macromer with 21% solid concentration in water was synthesized in house from Mowiol 3-83 (M_{w} 14,000 g/mol; Clariant, Charlotte, N.C., USA). PVA macromer contained 15 acrylamide groups per chain. Ammonium persulfate (APS), tetramethylethylenediamine (TMEDA), diacetone acrylamide (DAA), and crosslinkers such as methylenebisacrylamide (MBA), glycerol dimethacrylate (GD), bisphenol A ethoxylate diacrylate (BAED) were purchased from Sigma-Aldrich and used as received. Sodium chloride (NaCl), calcium chloride (CaCl₂), and potassium chloride (KCl) were purchased from VWR and Riedel-DeHaen respectively. Hydroxyapatite (HA) with different particle sizes was purchased from Berkeley Advanced Biomaterials Inc. Tricalcium phosphate (TCP) and amorphous calcium phosphate (ACP) were provided by nGimat Co. Ringer's solution, UPS water containing 8.6 g NaCl, 0.3 g KCl, and 0.33 g CaCl₂ per liter, was prepared in house.

Sample Preparation and Measurements

PVA macromer and DAA were mixed until the DAA was thoroughly dissolved. PVA/DAA, calcium salt, crosslinker and TMEDA were thoroughly mixed in an open mouth plastic container with a spatula, followed by the addition of APS, then mixing was continued for about 1 minute.

Axial compression testing: The mixed paste was quickly transferred into a cylindrical plastic mold with a diameter of 20 mm and a height of 9 mm, where it cured in about 10 minutes at room temperature. After curing, the samples were taken out and soaked in Ringer's solution at 37 C before testing. The axial compression testing was run on a MTS Insight 30 at a speed of 25 mm/minute at 37 C in Ringer's solution. The tangent modulus, ultimate stress, and ultimate strain were determined from the strain-stress curve of axial compression testing. All the data were based on at least 3 test specimens.

Calculations:

The calculation of volume percentage was based on following density numbers: PVA, water: 1.0; DAA: 0.998; HA: 3.15; TCP and ACP: 3.14; MBA: 1.235; GD: 1.12; BAED: 1.146.

The tangent modulus is the slope of the compression stress-strain curve at any specified stress or strain. Below the proportional limit the tangent modulus is equivalent to Young's modulus or elastic modulus. Tangent modulus at 1% or 10% strain were defined as the slope at 1% or 10% strain using a 2-order polynomial regression. Above the proportional limit the tangent modulus varies with strain and is most accurately found from test data. The ultimate point was defined at the point of maximum stress. The ultimate stress and strain are the stress and strain at the ultimate point.

TABLE 1
Mechanical Properties for Various Bone Cement Formulations

| | | | | | | | | | tangent | | | |
|---|---|---|---|---|---|---|---|---|---|---|---|---|
| | | | | | | Cross- | | Solid | Modulus | | | |
| | PVA | DAA | Water | Ca salt | | linker | | content | (MPa) at | Strength | Yield | Failure |
| ID | vol % | vol % | vol % | vol % | | vol % | | wt % | 1% strain | (MPa) | strain (%) | Mode** |
| 1 | 7.0 | 66.6 | 26.4 | 0 | | 0 | | 74 | 1.9* | 49 | 80 | No crack |
| 2 | 5.1 | 48.7 | 22.9 | 23.3 | (TCP) | 0 | | 85 | 5.7 | 90 | 80 | Crack |
| 3 | 4.5 | 42.5 | 32.8 | 20.3 | (ACP) | 0 | | 84 | 20 | 91 | 80 | Crack |
| 4 | 5.1 | 48.7 | 22.9 | 23.2 | (HA100 nm) | 0 | | 85 | 13 | — | — | No crack |
| 5 | 5.1 | 48.7 | 22.9 | 23.2 | (HA5 μm) | 0 | | 85 | 14 | 89 | 76 | Crack |
| 6 | 4.8 | 45.6 | 22.9 | 27.7 | (HA5 μm) | 0 | | 87 | 24 | 86 | 60 | Crack |
| 7 | 4.6 | 43.3 | 21.2 | 31.0 | (HA5 μm) | 0 | | 87 | 25 | 89 | 60 | Crack |
| 8 | 0 | 51.0 | 28.7 | 20.3 | (HA100 nm) | 0 | | 90 | 18 | — | — | Crack |
| 9 | 4.4 | 41.4 | 32.0 | 19.8 | (HA5 μm) | 2.5 | (MBA) | 88 | 94 | 63 | 37 | 1 piece |
| 10 | 4.2 | 40.4 | 31.1 | 19.3 | (HA5 μm) | 5.0 | (MBA) | 88 | 159 | 67 | 29 | 1 piece |
| 11 | 4.8 | 45.8 | 21.5 | 21.8 | (HA5 μm) | 6.0 | (BAED) | 75 | 75 | 19 | 30 | 1 piece |
| 12 | 4.3 | 40.4 | 19.8 | 28.9 | (HA5 μm) | 6.6 | (BAED) | 78 | 164 | 34 | 30 | 1 piece |
| 13 | 5.0 | 47.5 | 21.8 | 15.1 | (HA5 μm) | 10.6 | (DG) | 75 | 135 | 31 | 30 | 1 piece |
| 14 | 4.5 | 43.1 | 20.3 | 20.6 | (HA5 μm) | 11.6 | (DG) | 86 | 245 | 47 | 33 | 1 piece |
| 15 | 4.0 | 37.8 | 18.5 | 27.0 | (HA5 μm) | 12.7 | (DG) | 88 | 376 | 53 | 25 | shard |
| 16 | 6.8 | 32.3 | 28.8 | 20.5 | (HA5 μm) | 11.6 | (DG) | 71 | 89 | 21 | 29 | 1 piece |
| 17 | 6.0 | 28.3 | 26.0 | 27.0 | (HA5 μm) | 12.7 | (DG) | 75 | 144 | 32 | 19 | 1 piece |
| 18 | 0 | 48.8 | 16.0 | 29.1 | (HA5 μm) | 6.2 | (MBA) | 90 | 234 | 73 | 31 | 1 piece |

*Modulus at 10% strain.
**Crack- no failure and crack only; 1 piece- failed but stayed in 1 piece; Shard- failed into shards

### EXAMPLE 1

### PVA Hydrogels without Fillers

Table 1 summarizes the mechanical properties for the different formulations. As shown in Table 1, number 1, the mechanical properties of hydrogel itself (without inorganic filler) are low. The increase in crosslinking density, solid content can only slightly improve the mechanical properties, especially the modulus of the hydrogel system. An increase in crosslinking density or the concentration of DAA can increase the toughness of the hydrogel, but it will still be far from the stiffness and strength of vertebral cancellous bones and not suited for load bearing applications.

### EXAMPLE 2

### Addition of Fillers

In order to increase the modulus and strength of the hydrogel system, different calcium salts were explored systematically (see Table 1 samples 2-8). The salts were hydroxyapatite (HA), tricalcium phosphate (TCP), and amorphous calcium phosphate (ACP). The tangent modulus at 1% strain with different types of calcium salts ranges from about 5 to 25 MPa, which is up to 10 times higher than without filler. Calcium salts are very effective at increasing the mechanical properties of the formulations, e.g. the tangent modulus almost doubles when the HA concentration is increased from 49% to 58% (see samples 2 and 3). Despite the increase in tangent modulus, formulations with different calcium salts are surprisingly tough and did not fail when compressed up to 80% strain. The overall data indicates that the microscale HA is more effective in increasing the modulus compared to nanoscale HA. For formulations with calcium salts, the ultimate stresses are listed in Table 1, since they did not crack or only cracked, and there was no functional failure when tested up to 80% strain.

### EXAMPLE 3

### Addition of Fillers and Additional Crosslinkers

These bone cement formulations with different types of calcium salts can match cancellous bones with relatively low mechanical properties. Although further increases in the concentration of calcium salts can further increase the tangent modulus and the strength of the bone substitute, the increase in viscosity due to high concentration of calcium salts will induce additional problems in mixing and delivery for practical application. To overcome this problem, low molecular weight crosslinkers were introduced (Table 1, samples 9 to 18). The introduction of 5 to 10 wt % (2.5 to 13 vol %) crosslinker increased the tangent modulus up to about 370 MPa.

In general, crosslinkers are more effective in increasing the elastic modulus of the formulations compared to the effect of calcium salts. All bone cement formulations with crosslinkers are more brittle. As shown in Table 1, all samples with additional crosslinkers failed at much lower strain (20-40% axial compression) than without crosslinker. This has to be expected since the crosslinkers increase the crosslink density mainly in the DAA phase. Despite the increase in brittleness, these formulations are still much tougher than human vertebral cancellous bones, which have a failure strain at less than 5%.

The formulations with crosslinkers are also flexible in matching different bone types or degenerations. Formulations with elastic modulus from 90-240 MPa are available by simply decreasing the PVA and DAA concentration without a change in calcium salt content. The strength and failure strain are about 20-75 MPa and 20-40% respectively. These bone substitute formulations with different crosslinkers can match cancellous bones with medium and high mechanical properties.

In summary, a series of strong and tough bone substitute formulations have been developed. The mechanical properties of these formulations cover those of a range of vertebral cancellous bones.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

1. A bone substitute comprising a aqueous phase, an amphiphilic monomer that forms a hydrophobic phase, and an inorganic filler.

2. The bone substitute of claim 1, wherein the aqueous phase is a hydrogel phase formed by crosslinked macromers.

3. The bone substitute of claim 2, wherein the macromers have a backbone of a polymer comprising units with a 1,2-diol and/or 1,3-diol structure and at least two pendant chains including a crosslinkable group.

4. The bone substitute of claim 1, wherein the aqueous phase comprises 10 to 40% by volume of the bone substitute.

5. The bone substitute of claim 1, wherein the inorganic filler is present in an amount ranging from about 10 to 40 volume percent.

6. The bone substitute of claim 1, wherein the inorganic filler is present in an amount ranging from about 15 to 30 volume percent.

7. The bone substitute of claim 1, wherein the inorganic filler is calcium phosphate selected from the group monocalcium phosphate monohydrate (MCPM), dicalcium phosphate (DCP), tricalcium phosphate (TCP), amorphous calcium phosphate (ACP), hydroxyapatite (HA), tetracalcium phosphate (tetCP), and combinations thereof.

8. The bone substitute of claim 7, wherein the calcium phosphate has a size in the range of 0.001 to 500 μm.

9. The bone substitute of claim 1, wherein the amphiphilic monomer is diacetone acrylamide present in an amount from about 20 to 70 volume percent.

10. The bone substitute of claim 1, further comprising an additional crosslinker that has at least 2 reactive groups that react with the monomer or itself, and the molecular weight of the crosslinker is in the range of 30 to 5000.

11. The bone substitute of claim 2, wherein the bone substitute is formed in vivo by crosslinking after delivery.

12. The bone substitute of claim 1, wherein the tangent modulus is between about 5 and 800 MPa at 1% strain.

13. The bone substitute of claim 1, wherein the tangent modulus is between about 50 and 500 MPa at 1% strain.

14. The bone substitute of claim 1, wherein the ultimate stress is between about 0.5 and 30 MPa.

15. The bone substitute of claim 1, wherein the ultimate stress is between about 5 and 30 MPa.

16. The bone substitute of claim 3, wherein the macromer backbone is further modified with a hydrophobic or hydrophilic modifier.

17. The bone substitute of claim 1, wherein the bone substitute has a soft response to stress.

18. The bone substitute of claim 1, wherein the strain versus stress curve of the bone substitute nearly matches the strain versus stress curve of a cancellous bone.

19. A composition for use in implanting a bone substitute in vivo in a bone, comprising crosslinkable macromers, an amphiphilic monomer, and an inorganic filler,

20. The composition of claim 19, wherein the composition is injectable through an 8 to 13 gauge 4 to 6 inch long needle or cannula.

21. The composition of claim 19, wherein the proportions of the components are selected so that the bone substitute approximately matches the characteristics of the bone into which it is implanted.
